(19) United States
(12) Patent Application Publication
(10) Pub. No.: US 2009/0227486 A1
(43) Pub. Date: Sep. 10, 2009

(54) ENCAPSULATED PARTICLES

(76) Inventors: Fabrizio Meli, Tynemouth (GB); Paul Andrzej Luksza, Newcastle-upon-Tyne (GB); Keith James Stokoe, Newcastle-upon-Tyne (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/468,445

(22) Filed: May 19, 2009

Related U.S. Application Data
(63) Continuation of application No. 12/154,111, filed on May 20, 2008.
Application No. 12/154,111 is a continuation of application No. 11/142,949, filed on Jun. 2, 2005, now abandoned.

(30) Foreign Application Priority Data
Jun. 4, 2004 (EP) 04253367.9

(51) Int. Cl. A61K 8/11; A61Q 13/00; B01J 13/04

(52) U.S. Cl. 512/4; 264/4.1

(57) ABSTRACT

The present invention relates to a method for making starch encapsulated ingredients where a mixture of starch, water, acid and ingredient for encapsulation is prepared. The mixture is atomised and dried to provide encapsulates that can retain high levels of encapsulated ingredient. When the encapsulated ingredient comprises oil, the invention reduces the levels of free oil on the outside of the encapsulates and surprisingly reduces explosivity of fines produced during the manufacturing process. The preferred acid is citric acid.

### CROSS REFERENCE TO RELATED APPLICATIONS

This application is a continuation of and claims priority under 35 U.S.C. §120 to U.S. patent application Ser. No. 12/154,111, filed May 20, 2008, which in turn is a continuation of U.S. patent application Ser. No. 11/142,949, filed Jun. 2, 2005 (P&G Case CM2867).

### TECHNICAL FIELD

The present invention relates to the field of starch encapsulation. It relates to particles comprising encapsulated ingredients, methods for making them, compositions containing them and uses for such particles.

### BACKGROUND OF THE INVENTION AND PRIOR ART

Encapsulating specific ingredients in a starch-based encapsulate is well known where it is desired to form a water-soluble barrier between the component and its environment. The encapsulation is usually to protect a sensitive ingredient from its environment, or vice versa. For example in some compositions such as detergent compositions, one or more components may be sensitive to the atmosphere and/or the detergent matrix and encapsulation can therefore be used to protect such components during storage, prior to entry into the wash water.

Furthermore, most consumers have come to expect perfumed detergent products and to expect that fabrics and other items which have been laundered with these products also have a pleasing fragrance. However, some perfume ingredients are not stable on storage so that they need to be protected on storage as described above. In addition, for perfumes there is an additional factor that consumers do not like to be overwhelmed by strong perfume odours on opening the box or other container for detergent products. In order to provide sufficient odour on laundered fabrics, a relatively high amount of perfume is needed in a laundry product. Even then, since considerable dilution of the detergent occurs, there may be very limited fragrance on the laundered clothes. However, high loadings of perfume tend to make unacceptably strong odour for the detergent product itself. Encapsulation has therefore developed as a way of introducing more perfume into a product where it is desired that the product itself should not have a very strong odour.

Other examples of such products are any product where a subtle odour and/or flavour is required in the neat product and a stronger odour and/or flavour on contact of the product with water, such as in flavoured foods, bar soaps, paper products for use in the home such as towels, fragranced dryer sheets, etc. Other applications of starch encapsulation include encapsulation of pharmaceuticals and/or vitamins, where the encapsulation may be used to protect the pharmaceutical/vitamin and/or may be beneficial in making unpleasant-tasting drugs more palatable. The invention may also be used to encapsulate ingredients in the fields of personal care including hair care, papers products, animal care and household products. For example, other components besides perfumes suitable for encapsulation include silicone oils, waxes, hydrocarbons, higher fatty acids, essential oils, lipids, skin coolants, sunscreens, glycerine, catalysts, bleach particles, silicon dioxide particles, malodour reducing agents, antiperspirant actives, cationic polymers and mixtures thereof.

Examples of starch encapsulation are disclosed for example in WO 99/55819, WO 01/40430, EP-A-858828, EP-A-1160311 and U.S. Pat. No. 5,955,419. However, starch encapsulates such as those described in these applications are limited: relatively large amounts of starch have to be used for encapsulation and in addition, in the case of encapsulating oils such as perfume oils, some free oil is always present on the outside of the encapsulate particles.

The present inventors have found that it is possible to alleviate these problems and to prepare an encapsulated ingredient using lower amounts of starch. The present invention provides the additional benefit that where the encapsulated material comprises free oil, this invention reduces the free oil on the outside of encapsulates. This is particularly beneficial in the encapsulation of perfume oils as it enables incorporation of higher levels of perfume into products such as detergent compositions without increasing the dry odour of the detergent composition.

One other problem that may be associated with manufacture of starch encapsulates is related to the production of finely particulate material during manufacture. Since these materials are flammable, a build up of very fine particles may be explosive in the presence of oxygen and a source of ignition such as a spark. The present invention has surprisingly been found to have a considerable impact on reducing this problem.

### DEFINITION OF THE INVENTION

In accordance with the present invention there is provided a method for making an encapsulated ingredient comprising (a) preparing a mixture comprising starch, water, acid and ingredient for encapsulation, the acid being incorporated in the mixture in an amount sufficient to lower the pH of the starch-water mixture by at least 0.25 units; and (b) atomising and drying the mixture thereby forming encapsulated ingredient.

In accordance with further aspects of this invention there are provided encapsulated ingredients obtainable by the out-lined process and products containing such encapsulated ingredients.

All percentages, ratios and proportions herein are on a weight basis unless otherwise indicated. All documents cited are hereby incorporated by reference in their entirety.

### DETAILED DESCRIPTION OF THE INVENTION

In the first step in the process of the present invention an aqueous mixture is prepared comprising starch, water, ingredient for encapsulation and acid. These ingredients may be added in any order, but usually the starch-water mixture is prepared first and subsequently, either sequentially or together, the acid and ingredient for encapsulation are added. When they are added sequentially, the acid may be added prior to the ingredient for encapsulation. Alternatively, the acid is added after the ingredient for encapsulation.

The concentration of starch in the aqueous mixture may be from as low as 5 or 10 wt % to as high as 60 or even 75 wt %. Generally the concentration of starch in the mixture is from 20 to 50 wt %, more usually around 25 to 40 wt % in the aqueous mixture.

If the concentration is very low, in order to make the encapsulated particles obtained in the present invention, the energy cost to the process is high because of the need to remove high levels of water. The limiting factor on the upper concentration limit is the need to be able to process the mixture. Higher levels of starch can be accommodated as long as the mixture can still be atomised and dried to make finished product encapsulates. Other additives may be incorporated to reduce viscosity of the starch/water mixture and improve ease of handling. Suitable examples include emulsifiers and plasticisers.

Starches suitable for use in this first step can be made from raw starch, pregelatinized starch, modified starch derived from tubers, legumes, cereal and grains for example corn starch, wheat starch, rice starch, waxy corn starch, oat starch, cassava starch, waxy barley starch, waxy rice starch, sweet rice starch, amioca, potato starch, tapioca starch and mixtures thereof.

Modified starches may be particularly suitable for use in the present invention, and these include hydrolyzed starch, acid thinned starch, starch having hydrophobic groups, such as starch esters of long chain hydrocarbons (C5 or greater), starch acetates, starch octenyl succinate and mixtures thereof. Starch esters, particularly starch octenyl succinates are especially preferred.

The term “hydrolyzed starch” refers to oligosaccharide-type materials that are typically obtained by acid and/or enzymatic hydrolysis of starches, preferably corn starch. It may be preferred to include in the starch water-mixture, a starch ester. The hydrolyzed starches preferred, particularly for starch ester or mixture of starch esters, preferably have Dextrose Equivalent (DE) values of from 20 to 80, more preferably from 20 to 50, or even 25 to 38 DE. The DE value is a measure of the reducing equivalence of the hydrolyzed starch referenced to dextrose and expressed as a percent (on a dry basis). The higher the DE value, the more reducing sugars present. A method for determining DE values can be found in Standard Analytical Methods of the Member Companies of Corn Industries Research Foundation, 6th ed. Corn Refineries Association, Inc. Washington, D.C. 1980, D-52.

Particularly preferred starches are those wherein the starch is gelatinised and the hydrophobic group comprises an alkyl, or an alkenyl group which contains at least five carbon atoms or an aralkyl or aralkenyl group which contains at least six carbon atoms. Preferred starches for use in the present invention are starch esters. These will typically have a degree of substitution in the range of from 0.01% to 10%. The hydrocarbon part of the modifying ester should preferably be a C₅ to a C₁₋₆ carbon chain. As stated above octenyl succinate is the preferred ester. Preferably, octenyl succinate (OSAN) substituted waxy corn starches of various types such as 1) waxy starch, acid thinned and OSAN substituted, (2) blend of corn syrup solids: waxy starch, OSAN substituted and dextrinized, 3) waxy starch: OSAN substituted and dextrinised, 4) blend of corn syrup solids or maltodextrins with waxy starch: acid thinned OSAN substituted then cooked and spray dried, 5) waxy starch: acid thinned OSAN substituted then cooked and spray dried; and 6) the high and low viscosities of the above modifications (based on the level of acid treatment) can also be used in the present invention. Mixtures of these, particularly mixtures of the high and low viscosity modified starches are also suitable.

Particularly preferred are the modified starches comprising a starch derivative containing a hydrophobic group or both a hydrophobic and a hydrophilic group which has been degraded by at least one enzyme capable of cleaving the 1,4 linkages of the starch molecule from the non-reducing ends to produce short chained saccharides to provide high oxidation resistance while maintaining substantially high molecular weight portions of the starch base. Such starches are described in EP-A-922 449.

The aqueous starch mixture may also include a plasticizer for the starch. Suitable examples include monosaccharides, disaccharides, oligosaccharides and maltodextrins, such as glucose, sucrose, sorbitol, gum arabic, guar gums and maltodextrin.

The acid used in the process of the invention may be any acid. Examples include sulphuric acid, nitric acid, hydrochloric acid, sulphamic acid and phosphonic acid. However, carboxylic organic acids are more highly preferred, particularly preferred are the organic acids comprising more than one carboxylic acid group. Examples of suitable organic acids include citric acid, tartaric acid, maleic acid, malic acid, succinic acid, sebacic acid, adipic acid, itaconic acid, acetic acid and ascorbic acid, etc. Saturated acids are more usually used in the present invention. Particularly preferred is citric acid.

The acid is added to lower the pH of the mixture. Generally the acid is added to lower the pH of the mixture by at least 0.25 pH units, preferably by at least 0.5 units, or even at least 1 or 1.5 or 2 pH units. At the concentrations used in this invention, preferred starches provide a pH with water of no greater than 4.0. Typically, acid is added to lower the pH of the starch-water mixture to a value of 3.5 or below, or even below 3 or even below pH 2.

The ingredient for encapsulation may be any of the ingredients mentioned above as suitable for encapsulation, either alone or in combination with one another or with fillers, carriers and/or solvents. The invention is particularly aimed at encapsulation of flavour and/or perfume components and/or detergent active ingredients. It is particularly suitable for encapsulating ingredients comprising an oil component. The invention is also suitable for encapsulating such components present in microcapsules for example as disclosed in WO 2004/016234.

Useful ingredients for encapsulation include materials selected from the group consisting of perfumes such as 3-(4-t-butylphenyl)-2-methyl propanal, 3-(4-t-butylphenyl)-propanal, 3-(4-isopropylphenyl)-2-methylpropanal, 3-(3,4-methylenedioxyphenyl)-2-methylpropanal, and 2,6-dimethyl-5-heptenal, alpha-damascone, beta-damascone, delta-damascone, damascenone (2-Buten-1-one, 1-(2,6,6-trimethyl-1,3-cyclohexadien-1-yl)), 6,7-dihydro-1,1,2,3,3-pentamethyl-4(5H)-indanone, methyl-7,3-dihydro-2H-1,5-benzodioxepine-3-one, 2-[2-(4-methyl-3-cyclohexenyl-1-yl)propyl]cyclopentan-2-one, 2-sec-butylcyclohexanone, and -dihydro ionone, linalool, ethyllinalool, tetrahydrolinalool, and dihydromyrcenol; silicone oils, waxes such as polyethylene waxes; hydrocarbons such as petrolatum; essential oils such as fish oils, jasmine, camphor, lavender; skin coolants such as menthol, methyl lactate; vitamins such as Vitamin A and E; sunscreens; glycerine; catalysts such as manganese catalysts or bleach catalysts; bleach particles such as perborates, percarbonates, peracids or bleach activators; silicon dioxide particles; antiperspirant actives; cationic polymers, such as ditallowoyl ethanol ester dimethyl ammonium chloride, and mixtures thereof. Suitable ingredients can be obtained from Givaudan of Mount Olive, N.J., USA, International Flavors & Fragrances of South Brunswick, N.J., USA, or Quest of Naarden, Netherlands.

Other examples of perfume materials that are suitable for encapsulating using the encapsulation method of the present invention are those described in WO99/55819 from page 3. Particularly preferred perfumes for encapsulation according to the present invention include the HIA perfumes mentioned in that patent application, particularly those having a boiling point determined at the normal standard pressure of about 760 mmHg of 275° C. or lower, an octanol/water partition coefficient P of about 2000 or higher and an odour detection threshold of less than or equal 50 parts per billion (ppb). The preferred perfume ingredient have logP of 2 or higher.

Following the formation of the aqueous mixture comprising starch, water, ingredient for encapsulation and acid, the mixture is mixed under high shear to form an emulsion or dispersion of ingredient for encapsulation in the aqueous starch solution. Where the ingredient for encapsulation is an oil, the mixing should be at sufficient shear and for sufficient time to result in oily droplets having a diameter no greater than 2 mm, preferably no greater than 1.5 mm and preferably no greater than 1 mm, as measured under a microscope.

Any suitable technique may then be used for the final stage of processing where the aqueous mixture including acid and ingredient for encapsulation is atomised and dried. Suitable techniques include, but are not limited to those known in the art including spray drying, extrusion, spray chilling/crystallisation methods, fluid bed coating and the use of phase transfer catalysts to promote interfacial polymerization. Spray efficiencies may be increased by methods known in the art, such as by using high drying towers, lightly oiling the chamber walls, or using preconditioned air in which the moisture has been substantially removed.

The activity (payload) of the finished encapsulated product can be above 40 wt %, or above 50 wt % or even above 60 wt % or above 62 wt % of the starch encapsulated active ingredient. When the encapsulated ingredient comprises an oily component, it has been found that in accordance with the present invention, these surprisingly high payloads are also associated with unusually low free oil on the outside of the encapsulate. Thus for an activity of a perfume oil such as orange oil of 60 wt %, in accordance with the present invention, the encapsulates may also have a free oil content (measurement method given below) of no more than 1%, preferably below 0.75, or even below 0.5 wt %.

Measurement Method for Free Oil

1 g of Starch Encapsulates comprising encapsulated oil component is placed in a 40 mL glass vial. Then 5 mL of Hexane and 5 mL of a solution of hexadecane in hexane [(0.3 mg/mL)] are added into the same vial. The sample is shaken gently by hand for 2 min and let stand for 20 min to allow the particle to settle, an aliquot is taken for injection into the GC. In case the solution is not clear after the 20 min, the solution can be filtered [through a 0.45 um PDVF disc]. The hexadecane solution is used as internal standard. The quantification is done by comparison with the response from a Reference solution of the encapsulated oil in hexane which also contains the internal standard. The Reference solution is prepared based on free oil expected so similar GC responses are obtained from samples and reference, [for levels of free oil <1% a 0.7 mg/mL solution can be used] a fresh solution is prepared each day.

The next stage is the formation of the encapsulates: the starch-water mixture is agitated and atomised in any conventional means, for example by being pumped to a spray drying tower and being atomised for example from a spinning disc reactor. The sprayed droplets are then dried, encapsulates resulting.

The residence time of the acid in the water-starch mixture prior to atomisation is generally at least 15 minutes and no more than 72 hours. More usually the residence time will be no more than 24 o 12 or even 1 hour.

Other known methods of manufacturing the starch encapsulates of the present invention include but are not limited to, fluid bed agglomeration, extrusion, cooling/crystallisation methods and the use of phase transfer catalysts to promote interfacial polymerisation.

It may be preferred to incorporate into the mixture prior to the atomisation and drying stages, emulsification components or systems. Modified starches having emulsifying and emulsion stabilizing capacity such as starch esters, particularly octenyl succinates have the ability to entrap ingredients for encapsulation, particularly perfume oil droplets in the emulsion due to the hydrophobic character of the starch modifying agent. The ingredient for encapsulation such as perfume oils or flavourings remain trapped in the modified starch until contacted with water, for example on dissolution of a laundry detergent in wash solution, due to thermodynamic factors i.e. hydrophobic interaction and stabilization of the emulsion because of steric hindrance.

Preferred starches are described in EP-A-922499, U.S. Pat. No. 4,977,252, U.S. Pat. No. 5,354,559 and U.S. Pat. No. 5,935,826.

The encapsulated particles may contain perfumes or other ingredients suitable for incorporation into detergent compositions. The encapsulated particles are then added into detergents in an amount to provide the desired concentration of encapsulated component in the final detergent, for example at levels up to 50 wt % or higher depending on the encapsulated component. Generally, the encapsulated component will be a specialised ingredient usually added in small amounts, for example perfume or bleach components, particularly catalyst components. These will usually be present in amounts of from 0.01 wt % based on the detergent composition to 20 wt %, or from 0.05 to 10 wt % or from 0.05 to 3.0% or 0.05 to 1 wt %. the encapsulated particles preferably have a size of from about 1 micron to about 1000 microns. The particle size is controlled by the size of the suspensed particles in the mixture that is atomised and dried and the conditions of the atomising and drying stages.

Optional Detergent Adjuncts

As described above, detergent compositions comprising the particles of the invention will comprise at least some of the usual detergent adjunct materials, such as agglomerates, extrudates, other spray dried particles having different composition to those of the invention, or dry added materials. Conventionally, surfactants are incorporated into agglomerates, extrudates or spray dried particles along with solid materials, usually builders, and these may be admixed with the encapsulated particles of the invention.

The detergent adjunct materials are typically selected from the group consisting of detersive surfactants, builders, polymeric co-builders, bleach, chelants, enzymes, anti-redeposition polymers, soil-release polymers, polymeric soil-dispersing and/or soil-suspending agents, dye-transfer inhibitors, fabric-integrity agents, suds suppressors, fabric-softeners, flocculants, perfumes, whitening agents, photobleach and combinations thereof.

The precise nature of these additional components, and levels of incorporation thereof will depend on the physical form of the composition or component, and the precise nature of the washing operation for which it is to be used.

A highly preferred adjunct component is a surfactant. Preferably, the detergent composition comprises one or more surfactants. Typically, the detergent composition comprises (by weight of the composition) from 0% to 50%, preferably from 5% and more preferably from 10 or even 15 wt % to 40%, or to 30%, or to 20% one or more surfactants. Preferred surfactants are anionic surfactants, non-ionic surfactants, cationic surfactants, zwitterionic surfactants, amphoteric surfactants, cationic surfactants and mixtures thereof.

Preferred anionic surfactants comprise one or more moieties selected from the group consisting of carbonate, phosphate, sulphate, sulphonate and mixtures thereof. Preferred anionic surfactants are C₈₋₁₈ alkyl sulphates and C₈₋₁₈ alkyl sulphonates. Suitable anionic surfactants incorporated alone or in mixtures in the compositions of the invention are also the C₈₋₁₈ alkyl sulphates and/or C₈₋₁₈ alkyl sulphonates optionally condensed with from 1 to 9 moles of C₁₋₄ alkylene oxide per mole of C₈₋₁₈ alkyl sulphate and/or C₈₋₁₈ alkyl sulphonate. The alkyl chain of the C₈₋₁₈ alkyl sulphates and/or C₈₋₁₈ alkyl sulphonates may be linear or branched, preferred branched alkyl chains comprise one or more branched moieties that are C₁₋₆ alkyl groups. Other preferred anionic surfactants are C₈₋₁₈ alkyl benzene sulphates and/or C₈₋₁₈ alkyl benzene sulphonates. The alkyl chain of the C₈₋₁₈ alkyl benzene sulphates and/or C₈₋₁₈ alkyl benzene sulphonates may be linear or branched, preferred branched alkyl chains comprise one or more branched moieties that are C₁₋₆ alkyl groups.

Other preferred anionic surfactants are selected from the group consisting of: C₈₋₁₈ alkenyl sulphates, C₈₋₁₈ alkenyl sulphonates, C₈₋₁₈ alkenyl benzene sulphates, C₈₋₁₈ alkenyl benzene sulphonates, C₈₋₁₈ alkyl di-methyl benzene sulphate, C₈₋₁₈ alkyl di-methyl benzene sulphonate, fatty acid ester sulphonates, di-alkyl sulphosuccinates, and combinations thereof. The anionic surfactants may be present in the salt form. For example, the anionic surfactant may be an alkali metal salt of one or more of the compounds selected from the group consisting of: C₈₋₁₈ alkyl sulphate, C₈₋₁₈ alkyl sulphonate, C₈₋₁₈ alkyl benzene sulphate, C₈-C₁₈ alkyl benzene sulphonate, and combinations thereof. Preferred alkali metals are sodium, potassium and mixtures thereof. Typically, the detergent composition comprises from 10% to 30 wt % anionic surfactant.

Preferred non-ionic surfactants are selected from the group consisting of: C₈₋₁₈ alcohols condensed with from 1 to 9 of C₁-C₄ alkylene oxide per mole of C₈₋₁₈ alcohol, C₈₋₁₈ alkyl N—C₁₋₄ alkyl glucamides, C₈₋₁₈ amido C₁₋₄ dimethyl amines, C₈₋₁₈ alkyl polyglycosides, glycerol monoethers, polyhydroxyamides, and combinations thereof. Typically the detergent compositions of the invention comprises from 0 to 15, preferably from 2 to 10 wt % non-ionic surfactant.

Preferred cationic surfactants are quaternary ammonium compounds. Preferred quaternary ammonium compounds comprise a mixture of long and short hydrocarbon chains, typically alkyl and/or hydroxyalkyl and/or alkoxylated alkyl chains. Typically, long hydrocarbon chains are C₈₋₁₈ alkyl chains and/or C₈₋₁₈ hydroxyalkyl chains and/or C₈₋₁₈ alkoxylated alkyl chains. Typically, short hydrocarbon chains are C₁₋₄ alkyl chains and/or C₁₋₄ hydroxyalkyl chains and/or C₁₋₄ alkoxylated alkyl chains. Typically, the detergent composition comprises (by weight of the composition) from 0% to 20% cationic surfactant.

Preferred zwitterionic surfactants comprise one or more quaternized nitrogen atoms and one or more moieties selected from the group consisting of: carbonate, phosphate, sulphate, sulphonate, and combinations thereof. Preferred zwitterionic surfactants are alkyl betaines. Other preferred zwitterionic surfactants are alkyl amine oxides. Catanionic surfactants which are complexes comprising a cationic surfactant and an anionic surfactant may also be included. Typically, the molar ratio of the cationic surfactant to anionic surfactant in the complex is greater than 1:1, so that the complex has a net positive charge.

A further preferred adjunct component is a builder. Preferably, the detergent composition comprises (by weight of the composition and on an anhydrous basis) from 5% to 50% builder. Preferred builders are selected from the group consisting of: inorganic phosphates and salts thereof, preferably orthophosphate, pyrophosphate, tri-poly-phosphate, alkali metal salts thereof, and combinations thereof; polycarboxylic acids and salts thereof, preferably citric acid, alkali metal salts of thereof, and combinations thereof; aluminosilicates, salts thereof, and combinations thereof, preferably amorphous aluminosilicates, crystalline aluminosilicates, mixed amorphous/crystalline aluminosilicates, alkali metal salts thereof, and combinations thereof, most preferably zeolite A, zeolite P, zeolite MAP, salts thereof, and combinations thereof; silicates such as layered silicates, salts thereof, and combinations thereof, preferably sodium layered silicate; and combinations thereof.

A preferred adjunct component is a bleaching agent. Preferably, the detergent composition comprises one or more bleaching agents. Typically, the composition comprises (by weight of the composition) from 1% to 50% of one or more bleaching agent. Preferred bleaching agents are selected from the group consisting of sources of peroxide, sources of peracid, bleach boosters, bleach catalysts, photo-bleaches, and combinations thereof. Preferred sources of peroxide are selected from the group consisting of: perborate monohydrate, perborate tetra-hydrate, percarbonate, salts thereof, and combinations thereof. Preferred sources of peracid are selected from the group consisting of: bleach activator typically with a peroxide source such as perborate or percarbonate, preformed peracids, and combinations thereof. Preferred bleach activators are selected from the group consisting of: oxy-benzene-sulphonate bleach activators, lactam bleach activators, imide bleach activators, and combinations thereof. A preferred source of peracid is tetra-acetyl ethylene diamine (TAED) and peroxide source such as percarbonate. Preferred oxy-benzene-sulphonate bleach activators are selected from the group consisting of: nonanoyl-oxy-benzene-sulphonate, 6-nonamido-caproyl-oxy-benzene-sulphonate, salts thereof, and combinations thereof. Preferred lactam bleach activators are acyl-caprolactams and/or acyl-valerolactams. A preferred imide bleach activator is N-nonanoyl-N-methyl-acetamide.

Preferred preformed peracids are selected from the group consisting of N,N-pthaloyl-amino-peroxycaproic acid, nonyl-amido-peroxyadipic acid, salts thereof, and combinations thereof. Preferably, the STW-composition comprises one or more sources of peroxide and one or more sources of peracid. Preferred bleach catalysts comprise one or more transition metal ions. Other preferred bleaching agents are di-acyl peroxides. Preferred bleach boosters are selected from the group consisting of: zwitterionic imines, anionic imine polyions, quaternary oxaziridinium salts, and combinations thereof. Highly preferred bleach boosters are selected from the group consisting of: aryliminium zwitterions, aryliminium polyions, and combinations thereof. Suitable bleach boosters are described in U.S. 360568, U.S. Pat. No. 5,360,569 and U.S. Pat. No. 5,370,826.

A preferred adjunct component is an anti-redeposition agent. Preferably, the detergent composition comprises one or more anti-redeposition agents. Preferred anti-redeposition agents are cellulosic polymeric components, most preferably carboxymethyl celluloses.

A preferred adjunct component is a chelant. Preferably, the detergent composition comprises one or more chelants. Preferably, the detergent composition comprises (by weight of the composition) from 0.01% to 10% chelant. Preferred chelants are selected from the group consisting of: hydroxyethane-dimethylene-phosphonic acid, ethylene diamine tetra(methylene phosphonic) acid, diethylene triamine pentacetate, ethylene diamine tetraacetate, diethylene triamine penta(methyl phosphonic) acid, ethylene diamine disuccinic acid, and combinations thereof.

A preferred adjunct component is a dye transfer inhibitor. Preferably, the detergent composition comprises one or more dye transfer inhibitors. Typically, dye transfer inhibitors are polymeric components that trap dye molecules and retain the dye molecules by suspending them in the wash liquor. Preferred dye transfer inhibitors are selected from the group consisting of: polyvinylpyrrolidones, polyvinylpyridine N-oxides, polyvinylpyrrolidone-polyvinylimidazole copolymers, and combinations thereof.

A preferred adjunct component is an enzyme. Preferably, the detergent composition comprises one or more enzymes. Preferred enzymes are selected from then group consisting of: amylases, arabinosidases, carbohydrases, cellulases, chondroitinases, cutinases, dextranases, esterases, β-glucanases, gluco-amylases, hyaluronidases, keratanases, laccases, ligninases, lipases, lipoxygenases, malanases, mannanases, oxidases, pectinases, pentosanases, peroxidases, phenoloxidases, phospholipases, proteases, pullulanases, reductases, tannases, transferases, xylanases, xyloglucanases, and combinations thereof. Preferred enzymes are selected from the group consisting of: amylases, carbohydrases, cellulases, lipases, proteases, and combinations thereof.

A preferred adjunct component is a fabric integrity agent. Preferably, the detergent composition comprises one or more fabric integrity agents. Typically, fabric integrity agents are polymeric components that deposit on the fabric surface and prevent fabric damage during the laundering process. Preferred fabric integrity agents are hydrophobically modified celluloses. These hydrophobically modified celluloses reduce fabric abrasion, enhance fibre-fibre interactions and reduce dye loss from the fabric. A preferred hydrophobically modified cellulose is described in WO99/14245. Other preferred fabric integrity agents are polymeric components and/or oligomeric components that are obtainable, preferably obtained, by a process comprising the step of condensing imidazole and epichlorhydrin.

A preferred adjunct component is a salt. Preferably, the detergent composition comprises one or more salts. The salts can act as alkalinity agents, buffers, builders, co-builders, encrustation inhibitors, fillers, pH regulators, stability agents, and combinations thereof. Typically, the detergent composition comprises (by weight of the composition) from 5% to 60% salt. Preferred salts are alkali metal salts of aluminate, carbonate, chloride, bicarbonate, nitrate, phosphate, silicate, sulphate, and combinations thereof. Other preferred salts are alkaline earth metal salts of aluminate, carbonate, chloride, bicarbonate, nitrate, phosphate, silicate, sulphate, and combinations thereof. Especially preferred salts are sodium sulphate, sodium carbonate, sodium bicarbonate, sodium silicate, sodium sulphate, and combinations thereof. Optionally, the alkali metal salts and/or alkaline earth metal salts may be anhydrous.

A preferred adjunct component is a soil release agent. Preferably, the detergent composition comprises one or more soil release agents. Typically, soil release agents are polymeric compounds that modify the fabric surface and prevent the redeposition of soil on the fabric. Preferred soil release agents are copolymers, preferably block copolymers, comprising one or more terephthalate unit. Preferred soil release agents are copolymers that are synthesised from dimethylterephthalate, 1,2-propyl glycol and methyl capped polyethyleneglycol. Other preferred soil release agents are anionically end capped polyesters.

A preferred adjunct component is a soil suspension agent. Preferably, the detergent composition comprises one or more soil suspension agents. Preferred soil suspension agents are polymeric polycarboxylates. Especially preferred are polymers derived from acrylic acid, polymers derived from maleic acid, and co-polymers derived from maleic acid and acrylic acid. In addition to their soil suspension properties, polymeric polycarboxylates are also useful co-builders for laundry detergents. Other preferred soil suspension agents are alkoxylated polyalkylene imines. Especially preferred alkoxylated polyalkylene imines are ethoxylated polyethylene imines, or ethoxylated-propoxylated polyethylene imine. Other preferred soil suspension agents are represented by the formula:

bis((C₂H₅O)(C₂H₄O)ₙ(CH₃)—N⁺—CₓH₂ₓ—N⁺—(CH₃)-bis((C₂H₄O)ₙ(C₂H₅O)),

wherein, n=from 10 to 50 and x=from 1 to 20. Optionally, the soil suspension agents represented by the above formula can be sulphated and/or sulphonated.

### Softening System

The detergent compositions of the invention may comprise softening agents for softening through the wash such as clay optionally also with flocculant and enzymes.

Further more specific description of suitable detergent components can be found in WO97/11151.

### EXAMPLES

The following are examples of the invention.

### Example 1

### Emulsion Preparation and Spray Drying to Form Encapsulated Perfume Particles

500 g of HiCap 100 modified starch (supplied by National Starch & Chemical) are dissolved into 1000 g of deionised water to produce a homogeneous solution. 40 g of anhydrous citric acid is added to the starch solution and the mixture agitated for 10 minutes to dissolve the citric acid. At this point, 600 g of perfume are added. High shear mixing is then carried out for 10 minutes at around 2000 rpm using a ARD-Barico high shear mixer to produce an emulsion.

The emulsion is then pumped into a spray drier using a peristaltic pump and then spray-dried in a Production Minor cocurrent spray dryer manufactured by Niro A/S. A rotary atomising disc type FS 1, also from Niro A/S, is used to atomise the slurry. The air inlet temperature in the spray drier is 200° C. and the outlet temperature 90° C. The disc speed is set at 28,500 rpm. The tower is stabilized at these conditions by spraying water for 30 minutes before spray drying the emulsion. The dried particles are collected after particle/air separation in a cyclone. The particles produced had a mean particle size of 35 microns.

The perfume particles produced are suitable for incorporating into the detergent compositions exemplified below. Levels of incorporation are generally from 0.01 to 10 wt % based on the total weight of the detergent composition.

| | A | B | C | D | E |
|---|---|---|---|---|---|
| Sodium linear C₁₁₋₁₃ alkylbenzene | 11% | 12% | 10% | 18% | 15% |
| sulfonate | | | | | |
| R₂N⁺(CH₃)₂(C₂H₄OH), wherein R₂ = | 0.6% | 1% | | | 0.6% |
| C₁₂₋₁₄ alkyl group | | | | | |
| Sodium C₁₂₋₁₈ linear alkyl sulfate | | 0.3% | 2% | 2% | |
| condensed with an average of 3 to 5 | | | | | |
| moles of ethylene oxide per mole of | | | | | |
| alkyl sulfate | | | | | |
| Mid chain methyl branched sodium | 1.4% | 1.2% | 1% | | |
| C₁₂₋C₁₄ linear alkyl sulfate | | | | | |
| Sodium C₁₂₋₁₈ linear alkyl sulfate | 0.7% | 0.5% | | | |
| C₁₂₋₁₈ linear alkyl ethoxylate | | 3% | 2% | | |
| condensed with an average of 3-9 | | | | | |
| moles of ethylene oxide per mole of | | | | | |
| alkyl alcohol | | | | | |
| Citric acid | 2% | 1.5% | | | 2% |
| Sodium tripolyphosphate | | | 20% | 25% | 22% |
| (anhydrous weight given) | | | | | |
| Sodium carboxymethyl cellulose | 0.2% | 0.2% | | 0.3% | |
| Sodium polyacrylate polymer | | 0.5% | 1% | | 0.7% |
| having a weight average molecular | | | | | |
| weight of from 3000 to 5000 | | | | | |
| Copolymer of maleic/acrylic acid, | 2.1% | 2.3% | 2.1% | 1.4% | 1.4% |
| having a weight average molecular | | | | | |
| weight of from 50,000 to 90,000, | | | | | |
| wherein the ratio of maleic to | | | | | |
| acrylic acid is from 1:3 to 1:4 | | | | | |
| (Sokalan CP5 from BASF) | | | | | |
| EDDS (ethylenediamine-N,N′- | 0.3% | 0.5% | 0.6% | 0.4% | 0.4% |
| disuccinic acid (S,S isomer) in the | | | | | |
| form of its sodium salt) | | | | | |
| Diethylene triamine pentaacetic | 0.2% | | 0.5% | 0.2% | 0.3% |
| acid | | | | | |
| HEDP (1,1-hydroxyethane | 0.5% | 1.0% | 1.0% | 0.7% | 0.7% |
| diphosphonic acid) | | | | | |
| Proteolytic enzyme having an | 0.2% | 0.2% | 0.5% | 0.4% | 0.3% |
| enzyme activity of from 15 mg/g to | | | | | |
| 70 mg/g | | | | | |
| Amyolitic enzyme having an | 0.2% | 0.2% | 0.3% | 0.4% | 0.3% |
| enzyme activity of from 25 mg/g to | | | | | |
| 50 mg/g | | | | | |
| Lipolytic enzyme having an enzyme | | 0.2% | 0.1% | | |
| activity of 5 mg/g to 25 mg/g | | | | | |
| Anhydrous sodium perborate | | | 20% | 5% | 8% |
| monohydrate | | | | | |
| Sodium percarbonate | 10% | 12% | | | |
| Magnesium sulfate | 0.1% | 0.2% | 0.2% | 0.1% | 0.1% |
| Nonanoyl oxybenzene sulfonate | | | | 2% | 1.2% |
| Tetraacetylethylenediamine | 3% | 4% | 2% | 0.6% | 0.8% |
| Brightener | 0.1% | 0.1% | 0.2% | 0.1% | 0.1% |
| Sodium carbonate | 10% | 10% | 10% | 19% | 22% |
| Sodium sulfate | 20% | 15% | 5% | 13% | 1% |
| Zeolite A | 23% | 22% | 8% | 6% | 18% |
| Sodium silicate (2.0 R) | | 0.2% | | 1% | 1% |
| Crystalline layered silicate | 3% | 5% | 10% | | |
| Photobleach | 0.002% | | | | |
| Polyethylene oxide having a weight | 2% | 1% | | | |
| average molecular weight from 100 | | | | | |
| to 10,000 | | | | | |
| Perfume spray-on | 0.2% | 0.5% | 0.25% | 0.1% | |
| Starch encapsulated perfume | 0.4% | 0.1% | 2% | 3% | 0.5% |
| Silicone based suds suppressor | 0.05% | 0.05% | | | 0.02% |
| Miscellaneous and moisture | To 100% | To 100% | To 100% | To 100% | To 100% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for making an encapsulated ingredient comprising
(a) preparing a mixture comprising starch, water, acid and an ingredient for encapsulation, the acid being incorporated in the mixture in an amount sufficient to lower the pH of the starch-water mixture by at least 0.25 units; and
(b) atomising and drying the mixture thereby forming encapsulated ingredient.

2. A process according to claim 1 in which the starch-water-acid mixture has a pH no greater than 4.5.

3. A process according to claim 2 in which the starch-water-acid mixture has a pH no greater than 4.

4. A process according to claim 1 in which the starch and water are present in the mixture such that the concentration of starch is from 10 to 50 wt %.

5. A process according to claim 1 in which the starch comprises a starch ester.

6. A process according to claim 1 in which the ingredient for encapsulation comprises a detergent active component.

7. A process according to claim 1 in which the ingredient for encapsulation comprises a perfume or flavour component.

8. A process according to claim 1 in which the acid comprises an organic carboxylic acid.

9. A process according to claim 8 in which the acid comprises citric acid.

10. A process according to claim 1 in which in step (b) the acid is added to provide a pH reduction in the water-starch mixture of at least 0.5 pH units.

11. A process according to claim 10 in which in step (b) the acid is added to provide a pH eduction in the water-starch mixture of at least 1.0 pH units.

12. A process according to claim 1 in which the acid and starch are both present in the aqueous mixture for no more than 72 hours prior to atomisation and drying of the mixture.

13. A process according to claim 12 in which the acid and starch are both present in the aqueous mixture for no more than 24 hours prior to atomisation and drying of the mixture.

14. An encapsulated ingredient produced by the process of claim 1.

15. An encapsulated ingredient according to claim 14 comprising at least 40 wt %, ingredient based on the weight of the encapsulated ingredient.

16. An encapsulated ingredient according to claim 15 comprising at least 60 wt % ingredient.

17. An encapsulated ingredient according to claim 14 in which the encapsulated ingredient comprises perfume oil.
